(19) United States
(12) Patent Application Publication
(10) Pub. No.: US 2006/0020289 A1
(43) Pub. Date: Jan. 26, 2006

(54) Biocompatible and bioabsorbable suture and clip material for surgical purposes

(75) Inventor: Marc Kuttler, Berlin (DE)

Correspondence Address: HAHN LOESER & PARKS, LLP, One GOJO Plaza, Suite 300, AKRON, OH, 44311-1076, US

(73) Assignee: BIOTRONIK VI Patent AG, Baar (CH)

(21) Appl. No.: 11/187,261

(22) Filed: Jul. 22, 2005

(30) Foreign Application Priority Data
Jul. 23, 2004 (DE) 102004 036 399.4

(51) Int. Cl. A61L 17/00

(52) U.S. Cl. 606/228

(57) ABSTRACT

The present invention concerns a suture and clip material for surgical purposes which at least in parts comprises a biodegradable magnesium alloy with the following proportions by weight of the alloy components: rare earth metals between 2.0 and 4.0% by weight, yttrium between 3.5 and 4.5% by weight, zirconium between 0.3 and 1.0% by weight, no aluminum or at most aluminum <0.01% by weight, and balance <0.5% by weight, in particular <0.3% by weight, wherein magnesium occupies the proportion by weight that remains to 100% by weight in the alloy.

### BACKGROUND OF THE INVENTION

The invention concerns a suture and clip material for surgical purposes, which is extremely biocompatible, which can remain in the wound as it is absorbed by the body and which, by virtue of its composition, improves protection from wound infections and promotes the healing process.

The term suture and clip material is used to denote any material which is used to hold together identical or different kinds of tissue in human beings or animals until they have sufficiently grown together. In that respect, the tissue can be held together by different kinds and structures such as for example by threads, wires or clips. In that connection, healthy tissue is as far as possible not to be detrimentally affected.

Suture and clip materials in permanent form, that is to say, which do not involve any breakdown in time, and also in bioabsorbable form, have already long been known and used. The previous solutions however suffer from many unresolved disadvantages.

Bioabsorbable suture material in wire and thread forms comprising magnesium have long been known but have, however, not become established in the past by virtue of technological difficulties. Mention may be made here in particular of considerable development of gas, irregular degradation characteristics and inadequate bending properties for making knots.

In earlier times, in particular catgut and collagen materials were used as bioabsorbable suture material in thread configuration. Nowadays, in particular, synthetically produced polymers are used. A disadvantage of the polymers however is, inter alia, limited biocompatibility. Degradation is effected by a decomposition process which is initiated by the body and which entails, inter alia, inflammation phenomena. Such suture material is often complicated and expensive to produce and sterilize.

In the meantime, to enhance the level of efficiency involved in joining tissue or closing off wounds, surgical clips or staples of permanent materials are also used. Besides the saving in time in regard to use, easier sterilization and ease of use, stapling also guarantees better adaptation and eversion of the edges of the wound, which leads to better healing and markedly less obtrusive scars. A disadvantage however, is that the legs of the clips generally tear traumatic passages into the tissue, which frequently results in inflammation. Upon subsequent removal of the clips tissue damage and thus the risk of inflammation is even more pronounced. That results, in the least critical case, in conspicuous scars which are due to the legs of the clips.

In order to reduce such effects, the shape and deformation of the clips has been optimized. Tissue damage and inflammation upon removal of the clips however still cannot be entirely eliminated.

In order also to be able to use the clip procedures for tissue areas which are at a greater depth and which, after healing, are no longer accessible, and to avoid general tissue damage and inflammation due to clip removal, and also to make subsequent wound care still more efficient, biodegradable clips or staples are being developed and tested at the present time. They are based on polymers, like also the thread-like suture material. Difficulties that they involve are mechanical strength and deformability when of a suitable size or the degradation characteristics they involve. A major challenge is also the inflammation phenomena and infections which such materials entail.

In the context of further development of biodegradable metals with which it is possible to avoid many of the disadvantages of the polymer solutions, mention is made of magnesium alloys which inter alia are also to be suitable for use for threads, wires or clips. The alloys proposed include proportions of rare earth metals and lithium. The alloy can contain yttrium in a proportion of between 0.01 and 7% by weight and aluminum in a proportion of between 0.01 and 16% by weight.

### SUMMARY OF THE INVENTION

An aspect of the present invention is to improve biodegradable suture and clip material for surgical purposes. In particular, the invention aims to provide that biocompatibility is improved and body-specific healing and defense mechanisms are supported.

That aspect is attained by the suture and clip material according to the invention for surgical purposes, having the features recited herein. The suture and clip material comprises at least in parts a biodegradable magnesium alloy with the following proportions by weight of the alloy components:
rare earth metals between 2.0 and 4.0% by weight,
yttrium between 3.5 and 4.5% by weight,
zirconium between 0.3 and 1.0% by weight, and
balance <0.5% by weight, in particular <0.3% by weight,
   wherein magnesium occupies the proportion by weight that remains to 100% by weight in the alloy. The above-mentioned magnesium alloys are distinguished over the magnesium alloys which are known hitherto, in that they have mechanical properties which are adequate for the area of use and they exhibit controlled breakdown behavior which keeps gas liberation effects upon breakdown down to a very low level, which can be tolerated for the desired use.

In addition, in the context of the use of such magnesium alloys in in-vivo and in-vitro tests, it was shown that those alloys and their breakdown products are extremely biocompatible. Surprisingly, it was found in that respect that the use of such magnesium alloys counteracts strong immunological or inflammation reactions on the part of the body. It was also possible, in in-vitro tests, to verify controlled cell growth, in particular in respect of smooth muscle cells and endothelium cells. Growths which are the source of severe scarring effects appear to be averted or greatly checked. The operative mechanism which forms the basis for the positive effects involved has hitherto not been discovered in detail.

Generally known effects and influences of magnesium on bodily functions, which is usually absorbed by way of nutrition, point to the assumption that such processes are also at least locally activated by the direct use of magnesium or the magnesium alloys and the decomposition products which are absorbed upon degradation.

It is known for example, that magnesium in the organism has a positive influence on wound healing, as it is necessary for anaerobic metabolism and promotes normal granulation of the connective tissue, that is to say also rapid healing of the tissue damage caused inter alia by the suture. It is also known, in regard to antimicrobial effects of magnesium, that the non-specific defense by way of the properdin system is effective only in the presence of magnesium and phagocytosis of bacteria by leucocytes experiences stimulation by magnesium. Accordingly, magnesium provides inter alia for combating infections by assisting or activating the body-specific immune system and also generally reduces susceptibility to infections. That helps, inter alia, to counteract infections in the suture region.

The collective term “rare earth metal” is used here to denote the elements scandium (atomic number 21), lanthanum (57) and the 14 elements following lanthanum, cerium (58), praseodymium (59), neodymium (60), promethium (61), samarium (62), europium (63), gadolinium (64), terbium (65), dysprosium (66), holmium (67), erbium (68), thulium (69), ytterbium (70) and lutetium (71), as lanthanides.

“Biodegradation” in the sense in accordance with the invention concerns hydrolytic, enzymatic and other metabolism-governed breakdown processes in the living organism which lead to gradual dissolution of at least large parts of the materials used. The term biocorrosion is frequently used synonymously. The term bioresorption additionally includes subsequent resorption of the breakdown products.

### DETAILED DESCRIPTION OF THE INVENTION

Preferably, the balance of the magnesium alloy contains no aluminum or the aluminum proportion is at most <0.01% by weight. It has been found in investigations in respect of aluminum-bearing magnesium alloys that forgoing aluminum or using a proportion below the specified limit of aluminum still markedly improves the biocompatible of the alloy. Thus aluminum appears to have a toxic effect which is a source of trouble in regard to the uses described herein.

It is further preferred for the magnesium alloy to have a proportion of neodymium of between 1.5 and 3.0% by weight. The presence of neodymium in the alloy seems to substantially improve the material properties, in particular workability to afford wires and so forth.

It is further preferred for the balance to contain the elements lithium and/or zinc. Those elements—if present in small proportions—evidently have a positive influence on the properties of the material, that is to say, they facilitate workability and promote favorable degradation characteristics on the part of the magnesium alloy. Preferably, a proportion of lithium in the alloy is between 0.15 and 0.2% by weight and a proportion of zinc in the alloy is between 0.004 and 0.2% by weight.

In accordance with a preferred variant, the specific composition of the magnesium alloy and its modification are predetermined such that decomposition starts immediately after use as suture or clip material and mechanical integrity is maintained until the holding or closure function is no longer required, at least in partial regions, by virtue of the tissue in question having grown together. That period of time should preferably be between 1 and 30 days, in particular between 3 and 14 days. The extent of the breakdown processes is dependent on the conditions prevailing at the location of use.

The degradation progress can be controlled, inter alia, by the thickness of material, temperature treatments, surface treatments, the precise composition of the alloy or by bioabsorbable surface coatings.

Preferably, the magnesium-bearing suture and clip material is present in the form of thread, thread braided from individual threads, wire or clips of differing thickness, cross-sections and lengths. Other embodiments for the described functionality can also be envisaged.

The material can be easily sterilized in particular by means of gamma or beta irradiation or also with alcohol solutions.

What is claimed is:

1. A suture and clip material for surgical purposes which at least in parts comprises a biodegradable magnesium alloy with the following proportions by weight of the alloy components:
rare earth metals between 2.0 and 4.0% by weight,
yttrium between 3.5 and 4.5% by weight,
zirconium between 0.3 and 1.0% by weight, and
balance <0.5% by weight, in particular <0.3% by weight,
wherein magnesium occupies the proportion by weight that remains to 100% by weight in the alloy.

2. A suture and clip material as set forth in claim 1, wherein there is a proportion of aluminum in the alloy less than 0.01% by weight.

3. A suture and clip material as set forth in claim 1, wherein the magnesium alloy contains a proportion of neodymium of between 1.5 and 3.0% by weight.

4. A suture and clip material as set forth in claim 1, wherein the balance contains lithium and a proportion of lithium in the alloy is between 0.15 and 0.2% by weight.

5. A suture and clip material as set forth in claim 1, wherein the balance contains zinc and a proportion of zinc in the alloy is between 0.004 and 0.2% by weight.

6. A suture and clip material as set forth in claim 1, wherein a degradation behavior on the basis of the specific composition of the magnesium alloy and further modification and treatments is predetermined, such that the mechanical integrity remains maintained for at least between 1 and 30 days.

7. A suture and clip material as set forth in claim 2, wherein a degradation behavior on the basis of the specific composition of the magnesium alloy and further modification and treatments is predetermined, such that the mechanical integrity remains maintained for at least between 1 and 30 days.

8. A suture and clip material as set forth in claim 3, wherein a degradation behavior on the basis of the specific composition of the magnesium alloy and further modification and treatments is predetermined, such that the mechanical integrity remains maintained for at least between 1 and 30 days.

9. A suture and clip material as set forth in claim 4, wherein a degradation behavior on the basis of the specific composition of the magnesium alloy and further modification and treatments is predetermined, such that the mechanical integrity remains maintained for at least between 1 and 30 days.

10. A suture and clip material as set forth in claim 5, wherein a degradation behavior on the basis of the specific composition of the magnesium alloy and further modification and treatments is predetermined, such that the mechanical integrity remains maintained for at least between 1 and 30 days.
